(19) United States
(12) Patent Application Publication
(10) Pub. No.: US 2005/0021239 A1
(43) Pub. Date: Jan. 27, 2005

(54) Interpretation

(75) Inventor: Martin Bill, Birmingham (GB)

Correspondence Address: MERCHANT & GOULD PC, P.O. BOX 2903, MINNEAPOLIS, MN, 55402-0903, US

(73) Assignee: The Secretary of state for the home department, Birmingham (GB)

(21) Appl. No.: 10/674,590

(22) Filed: Sep. 29, 2003

(30) Foreign Application Priority Data
Jul. 24, 2003 (GB) 0317335.8

(51) Int. Cl. G06F 19/00; G01N 33/48; G01N 33/50

(52) U.S. Cl. 702/20

(57) ABSTRACT

An improved method of comparing a first data set with a second data set is provided, particularly suited for data sets arising from the analysis of DNA samples, in which both first and second data set are compared with one another, the comparison occurring before any processing of the data has occurred. In this way an empirical analysis of the data is provided which does not introduce any of the errors or data omissions inherent in an interpretive approach to data analysis.

This invention is concerned with improvements in and relating to interpretation, particularly in the context of forensic science.

A wide variety of situations in forensic science call for the comparison of a data set with another data set with a view to establishing whether they match or not. Various processes are used to establish whether or not a match exists. However, for STR and SNP analysis in particular, the data sets compared are processed data sets. That is to say that data in the raw data set has been manipulated, adjusted or had data excluded in order to give the processed data set. Typically this means the data set has been processed to convert it into potential allele designations. Various reasons exist for doing this. Having performed this step, the second step consists of the evaluation of the strength of evidence, using an approximation of the putative source and an approximation of the between source variability of the trace material. This evaluation is concerned with how well does the trace material “fit” with the potential alternative sources?

The present invention has amongst its aims to significantly reduce and/or avoid the need for data processing. The present invention has amongst its aims to improve the speed with which comparisons between a data set and another data set can be made. The present invention has amongst its aims to retain all useful data for subsequent use. The present invention has amongst its aims to replace the binary, genotypic model with an impirical model to extract and model features directly from the raw data. The present invention has amongst its aims to significantly reduce the problems of binary decisions in allelic designation, and to provide a fully continuous approach for the inference of identity of source from DNA trace material.

According to a first aspect of the invention we provide a method of comparing a first data set with a second data set, wherein:
the first data set is obtained by taking a first sample, the first sample being formed of one or more components, the components differing from one another in terms of one or more characteristics, subjecting the first sample to a technique which provides a detectable indication of the different characteristics possessed by different components, taking a reading of the detectable indication, the reading forming the first data set;
the second data set is obtained by taking a second sample, the second sample being formed of one or more components, the components differing from one another in terms of one or more characteristics, subjecting the second sample to a technique which provides a detectable indication of the different characteristics possessed by different components, taking a reading of the detectable indication, the reading forming the second data set; and
comparing the reading of the detectable indication for the first sample with the reading of the detectable indication for the second sample.

Preferably the first and second data sets are DNA data sets. The first and second data sets may be forensic science information data sets.

The first and/or second sample may be a DNA sample. The sample may be from a single source or may be a mixture from one or more sources. One or more sources of the sample may be known. One or more sources of the sample may be unknown. The sample may be purified and/or cleaned and/or otherwise altered before being subjected to the technique.

The method may be repeated in relation to one or more samples beyond the first and second samples. The comparison may include one or more further samples beyond the first and second samples.

The components may be different loci. The components may be different short tandem repeats. The components may be different single nucleotide polymorphisms. A plurality of different components may be under consideration in relation to a sample. They may be considered simultaneously. Preferably the same components are considered between samples. The components may be standard components which are considered in such techniques. A sufficient number of components may be considered to provide discriminating power between samples.

The components may differ from one another in respect of a single characteristic, for instance position or length or size or identity. The components may differ from one another in terms of two or more characteristics, such as any two selected from position or length or size or identity. The components may differ in terms of their size, particularly for short tandem repeat components, where preferably the number of based is considered. The components may differ in terms of their identity, particularly for single nucleotide polymorphism components, where preferably the identity of a base is considered.

The technique to which the sample is subjected is preferably the same for each sample. The technique may be a separation based on mobility in a medium. The technique may employ electrophoresis.

The detectable indication may arise as a result of the component itself. The detectable indication may be due to the collection and/or positioning of a component of the sample at a position, for instance in a medium. The detectable indication may arise as a result of an indicator which becomes associated with the component due to the technique, such as a dye or other form of label. The detectable indication may indicate the position or length or size or identity of the characteristic and/or component, or more than one of these.

Preferably the detectable indication is read by an instrument. The detectable indication may provide an output itself and/or an output may be stimulated. The detectable indication may be exposed to a radiation, particularly light, source. Preferably the reading is the radiation, preferably light, detected. Preferably the reading includes information on the position of the reading, for instance relative to the medium and potentially a reference point thereon. Preferably the reading includes information on the quantity of detectable indication and/or characteristic and/or component. Preferably the reading includes information on the quantity of detectable indication and/or characteristic and/or component for one or more positions. The positions may be considered relative to the medium and potentially a reference point thereon.

The reading may be a continuous set of data, for instance a continuous profile. The reading may be form of a spread or a spectrum of values. The reading may be in the form of a non-discrete set of data. The reading may be continuous and/or a spread and/or a spectrum and/or non-discrete across a range and particularly a range of positions, more particularly a range of physical positions, such as those on an analysis medium. The reading may be in the form of numerical values, particularly of position and/or size and/or height and/or area.

Preferably the reading is not processed in any way. Preferably the reading consists of the combination of all the detectable indications arising from the sample. Preferably the reading is not expressed and/or presented and/or considered and/or stored and/or recorded in terms of allele position and/or allele size and/or allele length and/or allele identity and/or quantity of an allele, ideally including any expression thereof. Preferably the reading is not expressed and/or presented and/or considered and/or stored and/or recorded in terms of SNP position and/or SNP size and/or SNP identity and/or quantity of an SNP and/or intensity of SNP, ideally including any expression thereof.

Preferably the reading does not exclude any data relating to the detectable indication. Preferably the reading does not exclude extractable data relating to the detectable indication. Preferably the reading does not represent an interpretation of the detectable indication.

Preferably the reading is stored and/or recorded and/or entered into a database. Preferably a record of the reading is kept, ideally in totally unaltered and/or unprocessed form.

The comparison is preferably made between the reading for the first sample and for the second sample with the readings not processed in any way. Preferably the comparison is made without the readings being expressed and/or presented and/or considered and/or stored and/or recorded in terms of allele position and/or allele size and/or allele length and/or allele identity and/or quantity of an allele, ideally including any expression thereof. Preferably the comparison is made without the readings being expressed and/or presented and/or considered and/or stored and/or recorded in terms of SNP position and/or SNP size and/or SNP identity and/or quantity of an SNP and/or intensity of SNP, ideally including any expression thereof.

Preferably the comparison is made between readings which do not exclude any data relating to the detectable indication. Preferably the comparison is made between readings which do not exclude extractable data relating to the detectable indication. Preferably the comparison is made between readings which do not represent an interpretation of the detectable indication.

Preferably at least one of the readings is stored and/or recorded and/or entered into a database prior to comparison. Preferably at least one of the readings is subject to a record of the reading being kept, ideally in totally unaltered and/or unprocessed form, prior to a comparison being made.

The reading for the first sample and/or for the second sample may be stored and/or recorded and/or entered onto a database with the readings not processed in any way. Preferably the reading for the first sample and/or for the second sample may be stored and/or recorded and/or entered onto a database without the readings being expressed and/or presented and/or considered and/or stored and/or recorded in terms of allele position and/or allele size and/or allele length and/or allele identity and/or quantity of an allele, ideally including any expression thereof. Preferably the reading for the first sample and/or for the second sample may be stored and/or recorded and/or entered onto a database without the readings being expressed and/or presented and/or considered and/or stored and/or recorded in terms of SNP position and/or SNP size and/or SNP identity and/or quantity of an SNP and/or intensity of SNP, ideally including any expression thereof.

Preferably the reading for the first sample and/or for the second sample may be stored and/or recorded and/or entered onto a database without excluding any data relating to the detectable indication. Preferably the reading for the first sample and/or for the second sample may be stored and/or recorded and/or entered onto a database without excluding any extractable data relating to the detectable indication. Preferably the reading for the first sample and/or for the second sample may be stored and/or recorded and/or entered onto a database without being an interpretation of the detectable indication.

The comparison may be used to look for matches between the first data set and the second data set. A match may be taken as indicative of a link between the samples involved and/or one or more contributors thereto and/or the circumstances from which the samples arose. A match may be used to assist investigations by law enforcement authorities. A match may be used as evidence in legal proceedings.

The comparison process may involve consideration of:

$$\frac{Pr ( Data / HpData \& Other information )}{Pr ( Data / HdData \& Other information )}$$

where this is an expression of the probability of the raw data given one raw data situation, for instance a prosecution scenario, considered against the probability of the raw data given another raw data situation, for instance a defence scenario.

The comparison may involve the use of Gaussian Mixture models. The comparison may involve the fitting of one or more Gaussian distributions to the first and/or second data set. The fitting processing may involve an iterative process. The parameters used in the fitting process may be used as a template. The template may be used in the comparison of one or more other samples with one or more other samples.

The first aspect of the invention may include any of the features, options or possibilities set out elsewhere in this application.

According to a second aspect of the invention we provide a method of comparing a first DNA data set with a second DNA data set, wherein:
the first DNA data set is obtained by taking a first DNA sample, the first DNA sample including one or more DNA components, the DNA components differing from one another in terms of one or more characteristics, subjecting the first DNA sample to a technique including amplification and component separation to provide a detectable indication of the different characteristics possessed by different DNA components, taking a reading of the detectable indication, the reading forming the first DNA data set, making a record of the first DNA data set in the form of the reading;
the second DNA data set is obtained by taking a second DNA sample, the second DNA sample including one or more DNA components, the DNA components differing from one another in terms of one or more characteristics, subjecting the second DNA sample to a technique including amplification and component separation to provide a detectable indication of the different characteristics possessed by different DNA components, taking a reading of the detectable indication, the reading forming the second DNA data set; and
comparing the reading of the detectable indication for the first DNA sample with the reading of the detectable indication for the second DNA sample to establish whether the first and second DNA samples match one another according to one or more criteria.

The second aspect of the invention may include any of the features, options or possibilities set out elsewhere in this application.

According to a third aspect of the invention we provide a method of comparing a first data set with a second data set, wherein:
the first data set is obtained by taking a first sample, the first sample being formed of one or more components, the components differing from one another in terms of one or more characteristics, subjecting the first sample to a technique which provides a detectable indication of the different characteristics possessed by different components, taking a reading of the detectable indication, the reading forming the first data set;
the second data set is obtained by taking a second sample, the second being formed of one or more components, the components differing from one another in terms of one or more characteristics, that subjecting the second sample to a technique which provides a detectable indication of the different characteristics possessed by different components, taking a reading of the detectable indication, the reading forming the second data set; and
comparing the reading of the detectable indication for the first sample with the reading of the detectable indication for the second sample, the comparison being made empirically and/or without interpreting detectable indications and/or without interpreting the reading of the detectable indications and/or without interpreting the data sets.

The third aspect of the invention may include any features, options or possibilities set out elsewhere in this application.

Various embodiments of the invention will now be described, by way of example only, and with reference to the accompanying drawings, in which:

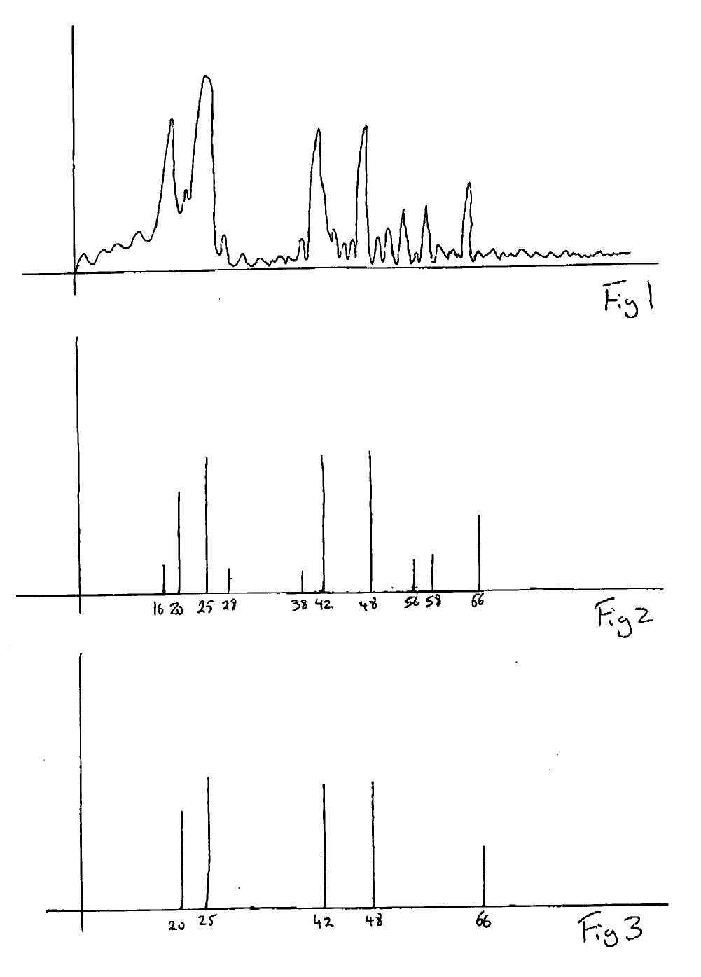

FIG. 1 is a schematic illustration of a raw data set occurring in STR analysis equipment;

FIG. 2 is a schematic illustration of the data set of FIG. 1 as a partially processed data set;

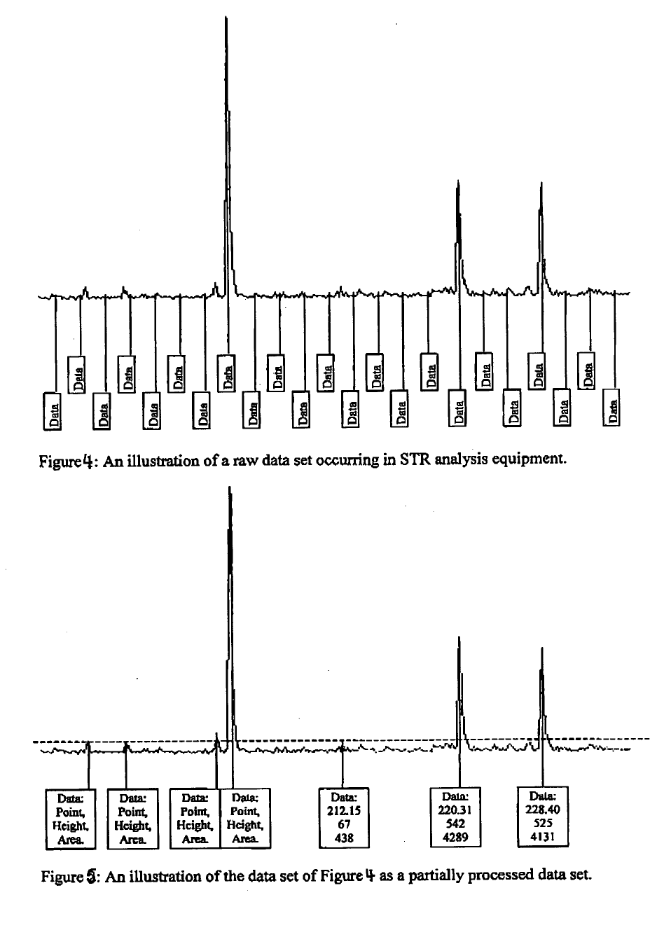

FIG. 3 is a schematic illustration of the processed data set derived from the data sets of FIGS. 1 and 2;

FIG. 4 is an illustration of a raw data set as it would actually occur in STR analysis equipment;

FIG. 5 is an illustration of the data set of FIG. 4 as a partially processed data set;

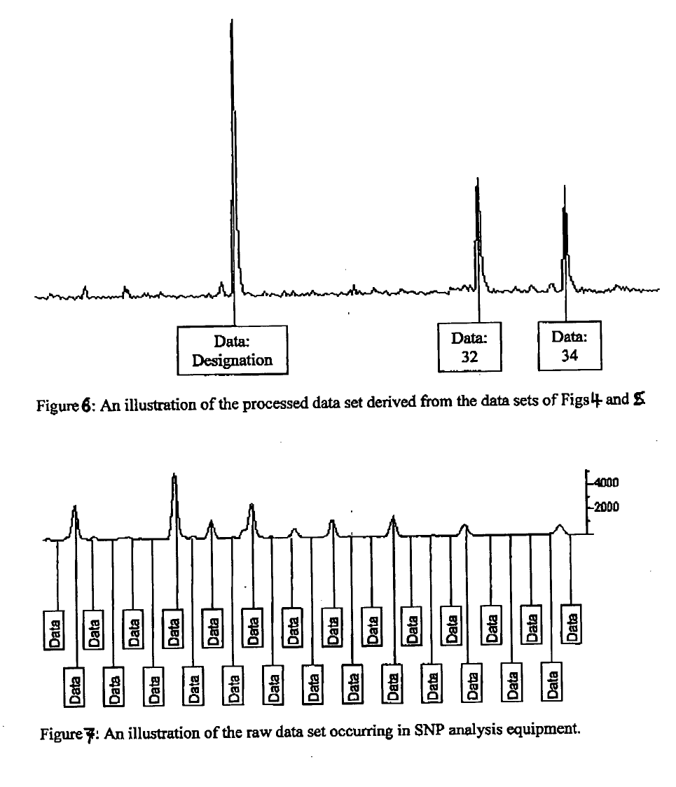

FIG. 6 is an illustration of the processed data set derived from the data sets of FIGS. 4 and 5;

FIG. 7 is an illustration of a raw data set as actually occurring in SNP analysis equipment;

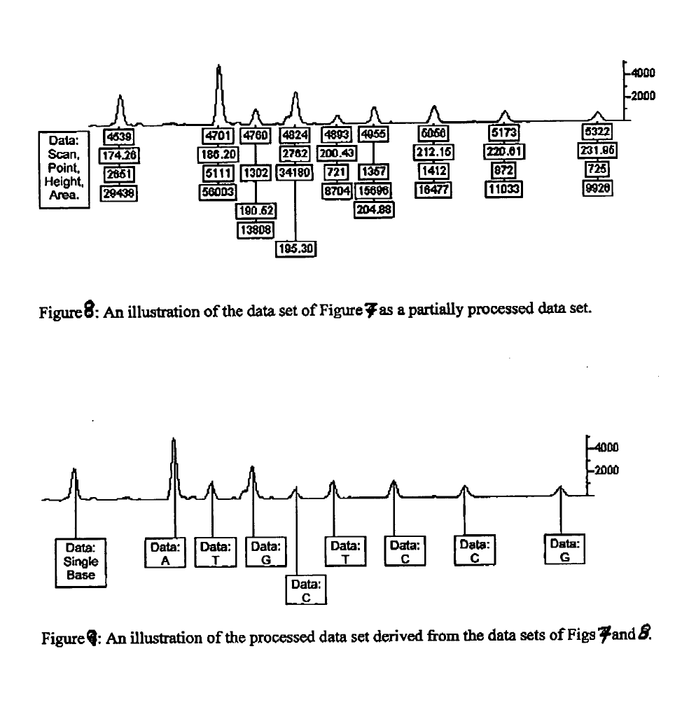

FIG. 8 is an illustration of the data set of FIG. 7 as a partially processed data set; and

FIG. 9 is an illustration of the processed data set derived from the data sets of FIGS. 7 and 8.

When considering DNA samples in the context of forensic science a number of stages are normally involved in the process. Firstly, a DNA sample is collected, for instance from a crime scene. The DNA sample may then be pre-treated to maximise the useful DNA for analysis, for instance through cleaning of the sample. The DNA sample is usually then amplified, for instance using PCR and is then introduced to an analysis equipment, such as a sequencer.

It is during its time in the analysis equipment that the processing of the raw data may first occur.

In the case of STR based analysis, the analysing equipment aims to produce as its output an indication of allele position and the peak height or peak area associated with that allele position, for each of the alleles detected.

To achieve this the analysis equipment performs the operation necessary to allow distinctions between the alleles to be drawn. In many cases, this represents a separation based on different mobilities within in a medium. The analysis equipment then inspects the medium to obtain a raw data set from it. This may involve determining the quantity of light reaching the analysis equipment in response to interogation using a laser, for instance. This generally involves a large, but obviously finite, number of interogations, each at a different position on the medium.

The analysis equipment then takes this raw data set and immediately subjects it to a processing stage. No record of the raw data set is made or stored. The processing aims to calculate allele positions and for each position a peak height or peak area value. This involves the analysis equipment extracting some data and discarding the rest of the raw data set. Whilst the raw data set could be represented graphically as a continuous style plot of the type shown in FIG. 1 the processed data set is formed only of the calculated allele positions which have peak heights/areas above a threshold level, FIG. 2. These positions are determined to be alleles that are present. A processed data set containing data of the FIG. 2 type is outputted by the analysis equipment. Generally, the output is a file containing this processed data set in numerical form. A permenant record of this processed data set is usually made. A key feature of the processed data set is that it is an expression of calculated identities (the particular alleles) and of quantity (the peak area/height information attributed to those alleles). The remaining data in the raw data set has been excluded and is not used further as it is accepted as being noise. This part of the processing occurs within the analysis equipment.

Having processed the raw data set in this way, the processed data set may be subjected to further processing. In many cases this is in the form of interpretation by an expert or expert system. According to certain rules and/or experience the interpretation stage may remove further parts of the data set from consideration. Allele positions which are deemed to be due to stutter and/or allele drop in and/or degradation etc may be excluded in this way. The result is that some of the peaks are discarded in reaching the final processed data set represented in FIG. 3. The end result of the processing is a processed data set which is taken as the DNA profile of the DNA sample. This processed data set can be loaded onto a database as a genotype. This further processing starts with a processed data set which considers its data elements in terms of alleles, interpretes them in that way and expresses its final results also in terms of alleles.

FIGS. 4, 5 and 6 illustrate raw, partially processed and fully processed data sets for STR analysis equipment in the format they would actually be observed in practice.

In the case of SNP based analysis, the analysing equipment aims to produce an indication of SNP identity, locus position and the peak height or peak area associated with that position, for each of the positions detected. A similar process is used to obtain the raw data set. Once this is done, the analysis equipment takes the data, which could be represented as a continuous style plot and processes the raw data set to extract a processed data set from its. In effect, the processed data set is formed only of the positions which have peak heights/areas above a threshold level again. The position is used to calculate the particular locus and the peak the particular base present at the SNP site, usually depending on the dye colour detected. Once again the processed data set is an expression in terms of calculated identities (the particular loci for which SNP's are considered and the base occuring at that site) and of quantity (the peak area/height attributed to that SNP). The remaining data of the raw data set is not used further as it is accepted as being noise. Again this part of the processing occurs within the SNP analysis equipment.

Again the processed data may be subjected to still further processing before reaching its final form. It is this processed data set which is taken as the profile, is loaded onto the database and is in effect the genotype against which searches are made. Generally, the data sets are handled in numerical, rather than graphical form.

FIGS. 7, 8 and 9 illustrate raw, partially processed and fully processed data sets for SNP analysis equipment in the format they would actually be observed in practice.

These forms of data sets can be thought of as applying to each sample considered. IN respect of a first sample, there is a raw data set which is partially processed within the analysis equipment to give a processed data set and then further processed, interpretation, to give a genotype. A similar approach is taken with other samples. The raw data set is presently an entirely transitory position within the analysis equipment and no record of it is kept. Present uses of a processed data set, a genotype, would include considering that genotype against other genotypes recorded on the database to look for matches. When looking for matches to date, the overwhelming approach used has been to compare a genotype, a processed data set, with another genotype, another processed data set.

It is this approach which is used in relation to existing databases, such as The National DNA Database (Reg TM), which stores processed data in the form of genotypes. With regard to these genotypes it is possible to search against the database for a match between two or more genotypes. The search genotype is based on processed data just as the contents of the database are. Matches can be used to give valuable information to assist in further law enforcement enquiries or to back up the likelihood of a suggested position.

In the subsequent use of the results the prior art concerns itself with the Bayesian proposition:

$$\frac{Pr ( Data / HpGenotype \& other information )}{Pr ( Data / HdGenotype \& other information )}$$

where Hp is the proposed prosecution genotype and Hd is the proposed defence alternative genotype.

In a far more limited number of cases, there has been some comparison of a partially processed data set with the fully processed data sets already on the database. This occurs in the procedure outlined in I. W Evett et al. Journal of Forensic Science, January 1998, pp62-69, “Taking Account of Peak Areas when Interpreting Mixed DNA Profiles”. Even in this case, however, allele positions and peak areas are extracted from the raw data set by the analysis equipment and it is the consideration of genotypes which is undertaken. The comparison process looks for alleles in the same position, between cases. The same approach to matching and to the subsequent use of the results is taken to that outlined above.

The problem with all the prior art approaches is that it is based on an interpretive method. The genotype of a suspect is never actually known. It is merely suggested based on the information to hand. However, this information is subject to an error rate, with potential errors arising from each processing stage due to the data discarded in it.

The fundamentally different approach of the present invention is based upon the direct consideration of the raw data set. This in turn leads to a different expression of the position, namely:

$$\frac{Pr ( Data / HpData \& Other information )}{Pr ( Data / HdData \& Other information )}$$

Thus it is the probability of the raw data given one raw data situation which is being considered against the probability of the raw data given another raw data situation.

The acceptance of this approach leads to the creation of a fundamentally different database from that presently used. The new form of database stores the data in unprocessed form. Thus the raw data sets obtained from the analysis equipment before any processing are loaded onto the database. There is no clean up, potentially no application of thresholds, no interpretation of the data or other processing of the data before loading into the database. Once on the database matching algorithms can be used to establish matches between raw data sets.

When optimising this system, the comparison run time may be a significant issue to overcome. In order to compensate for this a detection threshold may be applied, but in a manner which does not compromise the nature of the raw data set stored. By raising the threshold the information relating to the lower points on the trace will be omitted from the result. This will focus the interpretation on the higher, more pertinent areas. Crucially the result remains raw as there is no attempt to assign allele or artefactual scores to the data. The raw data set continues to be in the form of a series of point and height area data elements.

The ability to be able to load the raw data sets straight from the analysis equipment into the database and then perform an immediate search is highly beneficial in a number of ways.

Firstly, this sequence of operations is possible in very short period of time. This compares with the prior art where the need to process the raw data set to account for various issues, followed by its interpretation (often using an expert) to reach the processed data set means that a significant time delay arises before the data set can be loaded onto the database.

Furthermore, each of the “processes” and “interpretations” takes the data set and effectively filters it to remove some of the information. In most cases it is likely that this filter will remove from the data set some information which relates to the DNA under consideration, as well as the noise and other such non-informative data. Each step in the prior art involves data loss, whereas the present invention does not run this risk as all data is included in the raw data set which is loaded onto the database.

Because the approach considers raw data sets against one another, there is no need for experts to interpret the data set during its processing. This reduces the cost of the process significantly as automation of the entire process is possible.

Because all of the data collected is used in the comparison process, the error rate should also be reduced when compared with existing systems. No processing which could accidentally discard relevant information from the profile occurs in the present invention.

Because the approach does not require data sets to conform to a restrictive data format, e.g. the allele results for the suggested principal contributor to the sample, it will be possible to load a larger percentage of case samples to the database. This would increase the success rate and overall effectiveness of DNA profiling.

A database following such an approach is also more suited to handling future improvements as all the data is retained. Thus the information on the database is useful whatever new techniques are developed in the future.

Whilst the description of the invention above has been made in relation to the consideration of DNA samples, it is potentially applicable in a similar way to a very large range of data forms, particularly within the context of forensic science. The method of the invention could potentially be used to consider analysis of other situations, such as those including drugs, speech considerations, drug data and speaker data.

A variety of techniques could be used as the basis for comparing the complex signal which one raw data set represents with the complex signal an other raw data set represents. One such technique is Gaussian Mixture Models, GMM's. GMM's can be used to fit a set of Gaussian distributions to the raw data set. An iterative algorithm achieves the fitting process. Once obtained by such a fit, the group of parameters which define the fit can be used as a template and that in turn can be used to compare raw data sets in a database. The template can be used to compare both raw data sets from known samples and from unknown samples, without the need to establish designation of peaks etc.

1. A method of comparing a first data set with a second data set, wherein:
the first data set is obtained by taking a first sample, the first sample being formed of one or more components, the components differing from one another in terms of one or more characteristics, subjecting the first sample to a technique which provides a detectable indication of the different characteristics possessed by different components, taking a reading of the detectable indication, the reading forming the first data set;
the second data set is obtained by taking a second sample, the second sample being formed of one or more components, the components differing from one another in terms of one or more characteristics, subjecting the second sample to a technique which provides a detectable indication of the different characteristics possessed by different components, taking a reading of the detectable indication, the reading forming the second data set; and
comparing the reading of the detectable indication for the first sample with the reading of the detectable indication for the second sample.

2. A method of comparing a first DNA data set with a second DNA data set, wherein:
the first DNA data set is obtained by taking a first DNA sample, the first DNA sample including one or more DNA components, the DNA components differing from one another in terms of one or more characteristics, subjecting the first DNA sample to a technique including amplification and component separation to provide a detectable indication of the different characteristics possessed by different DNA components, taking a reading of the detectable indication, the reading forming the first DNA data set, making a record of the first DNA data set in the form of the reading;
the second DNA data set is obtained by taking a second DNA sample, the second DNA sample including one or more DNA components, the DNA components differing from one another in terms of one or more characteristics, subjecting the second DNA sample to a technique including amplification and component separation to provide a detectable indication of the different characteristics possessed by different DNA components, taking a reading of the detectable indication, the reading forming the second DNA data set; and
comparing the reading of the detectable indication for the first DNA sample with the reading of the detectable indication for the second DNA sample to establish whether the first and second DNA samples match one another according to one or more criteria.

3. A method according to claim 2 in which the reading is not processed in any way.

4. A method according to claim 2 in which the reading is not expressed and/or presented and/or considered and/or stored and/or recorded in terms of allele position and/or allele size and/or allele length and/or allele identity and/or quantity of an allele, ideally including any expression thereof.

5. A method according to claim 2 in which the reading is not expressed and/or presented and/or considered and/or stored and/or recorded in terms of SNP position and/or SNP size and/or SNP identity and/or quantity of an SNP and/or intensity of SNP, ideally including any expression thereof.

6. A method according to claim 2 in which the reading does not exclude any data relating to the detectable indication.

7. A method according to claim 2 in which the reading does not represent an interpretation of the detectable indication.

8. A method according to claim 2 in which at least one of the readings is stored and/or recorded and/or entered into a database prior to comparison.

9. A method according to claim 2 in which the comparison process involves consideration of:
Pr(Data/HpData & Other information)
Pr(Data/HdData & Other information)
where this is an expression of the probability of the raw data given one raw data situation, for instance a prosecution scenario, considered against the probability of the raw data given another raw data situation, for instance a defence scenario.

10. A method of comparing a first data set with a second data set, wherein:
the first data set is obtained by taking a first sample, the first sample being formed of one or more components, the components differing from one another in terms of one or more characteristics, subjecting the first sample to a technique which provides a detectable indication of the different characteristics possessed by different components, taking a reading of the detectable indication, the reading forming the first data set;
the second data set is obtained by taking a second sample, the second being formed of one or more components, the components differing from one another in terms of one or more characteristics, that subjecting the second sample to a technique which provides a detectable indication of the different characteristics possessed by different components, taking a reading of the detectable indication, the reading forming the second data set; and
comparing the reading of the detectable indication for the first sample with the reading of the detectable indication for the second sample, the comparison being made empirically and/or without interpreting detectable indications and/or without interpreting the reading of the detectable indications and/or without interpreting the data sets.
